(19) United States
(12) Patent Application Publication
(10) Pub. No.: US 2011/0301546 A1
(43) Pub. Date: Dec. 8, 2011

(54) HINGED CAP FOR NEEDLE DEVICE

(76) Inventors: Volker Harms, Kassel (DE); Juergen Fuchs, Bad Emstal (DE); Markus Kaemereit, Nottuln (DE); Frank-Michael Roediger, Burgdorf (DE)

(21) Appl. No.: 13/129,802

(22) PCT Filed: Oct. 28, 2009
(86) PCT No.: PCT/US09/62294

§ 371 Date: Aug. 25, 2011

Related U.S. Application Data
(60) Provisional application No. 61/115,664, filed on Nov. 18, 2008.

(51) Int. Cl. A61M 5/32

(52) U.S. Cl. 604/192

(57) ABSTRACT

A hinged cap device for use with a syringe includes a base defining an interior cavity for mounting to a tip, a cap connected to the base by a living hinge and an outer shell positioned over the cap. The hinged cap device is moveable from an unlocked position, wherein the cap is rotated away from the needle hub to expose a needle, to a temporary locked position, wherein the outer shell is latched onto the cap to prevent the cap from being displaced from the needle hub; and from the unlocked position to a permanently locked position, wherein the needle is secured within the cap. A temporary locking mechanism is on the outer shell to prevent the displacement of the cap from the needle. A permanent locking mechanism located on the cap and comprises a pin projected from a button lock of one side wall of the cap and a locking bore located on a second side wall of the cap, wherein the pin is engageable within the locking bore to secure the needle within the cap.

### CROSS-REFERENCE TO RELATED APPLICATIONS

This application is a national phase application of International Application No. PCT/US2009/062294, filed Oct. 28, 2009, entitled HINGED CAP FOR NEEDLE DEVICE, which claims the benefit of U.S. Provisional Application No. 61/115,664, filed Nov. 18, 2008, the contents of which are expressly incorporated herein by reference.

### FIELD OF ART

The present invention relates generally to caps for a needle device and, more particularly, to hinged cap devices for use with hypodermic needles.

### BACKGROUND

Recapping is a common procedure for periods between drawing up fluids into a syringe and administering injections through a needle. The recapping procedure can occasionally cause needle sticks since users sometime misalign the needles with the openings on the caps, producing minimal pain but causing a great deal of inconvenience because all such needle stick incidences must be reported. Also, since needles related to the needle stick incidences must be discarded, medications contained within the syringes are unnecessarily wasted. Furthermore, fluids linked to these “clean” types of needle sticks can cause injuries and adverse reactions.

In an effort to reduce or eliminate the source of “clean” needle stick injuries during recapping between drawing up fluids and administering an injection, it was necessary to improve the state of the art. The improvements will also minimize or eliminate the more dangerous types of needle stick injuries that occur after the needles have been contaminated with a patient's bodily fluids. In exemplary embodiments discussed below, hinged cap devices are packaged ready for use with or without additional removable caps.

### SUMMARY

According to the present invention, there is provided a hinged cap device for use with a syringe comprising a needle hub comprising a cavity for fitting onto a syringe tip, said needle hub comprising a needle comprising a needle shaft and a needle tip; a cap defining a channel for capturing the needle to shield the needle tip; a living hinge connecting the cap to the needle hub; a depressable button lock for locking the needle so that the needle is not displaceable from the cap; said depressable button lock being located on a proximal end of a first side wall of said cap and comprising a pin projecting from said first side wall towards a second side wall of said cap; and a lock receiving plate located on said second side wall of said cap at a location for receiving said pin.

According to another aspect of the present invention, there is provided a hinged cap device for use with a syringe comprising: a needle hub comprising a cavity for fining onto a syringe tip, said needle hub comprising a needle comprising a needle shaft and a needle tip; a cap defining a channel for capturing the needle to shield the needle tip; a living hinge connecting the cap to the needle hub; a locking mechanism on the cap for securing the needle within the cap, the locking mechanism comprising a pin projected from a button lock locatable on a first side wall of said cap; a locking bore located on a second side wall of said cap; and wherein the pin is engageable within the locking bore to secure the needle within the cap.

According to another aspect of the present invention, there is provided a method for making a hinged cap device for use with a syringe, said method comprising: forming a needle hub having a cavity for mounting on a syringe tip and attaching a needle to the needle hub; forming a primary cap having a base wall, a first side wall, and a second side wall, which together define a channel, and attaching the primary cap to the needle huh with a living hinge; rotating the primary cap so that the needle is captured within the channel defined by the primary cap; providing a temporary lock so that when the temporary lock is activated, the primary cap is temporarily blocked from being rotated away from the needle; providing a permanent lock so that when the permanent lock is activated, the primary cap is permanently blocked from being rotated away from the needle; and wherein the permanent lock is located on one of the two side walls and comprises an annular race defining a cavity, a push button comprising a push wall comprising a push surface movable into the cavity of the annular race, and a pin extending from the push wall and configured to extend across the channel of the primary cap when the permanent lock is activated to contact a lock receiving plate on the second side wall.

Other alternatives and embodiments of the hinged cap device in accordance with the present invention are also described herein and further discussed below in the Detailed Description section.

### BRIEF DESCRIPTION OF THE DRAWINGS

These and other features of advantages of the present invention will become appreciated as the same becomes better understood with reference to the specification, claims and appended drawings wherein:

FIG. 1 is a side view of a hinged cap device provided in accordance with aspects of the present invention mounted on a syringe;

FIG. 2 is a perspective view of the hinged cap device of FIG. 1, showing the cap rotated away from the needle hub to expose the needle;

FIG. 3 is another perspective view of the hinged cap device of FIG. 1, showing a notch on the outer shell interacting with a gripping lever on the cap to latch the outer shell onto the cap;

FIG. 4 is a perspective view of the hinged cap device of FIG. 1 in an unlocked position, showing the outer shell removed from the cap and the cap rotated away from the needle hub to expose the needle holder;

FIG. 5 is a cross-sectional top view of the hinged cap device of FIG. 4 in an unlocked position;

FIG. 6 is another cross-sectional top view of the hinged cap device of FIG. 4 in a locked position, showing a pin engaging in a lock receiving plate of a lock mechanism;

FIG. 7 is a perspective view of another exemplary hinged cap device provided in accordance with aspects of the present invention;

FIG. 8 is a side view of the hinged cap device of FIG. 7, showing the cap positioned over the needle to shield the needle but without the outer shield;

FIG. 9 is a side view of the outer cap of FIG. 7 removed from the inner cap;

FIG. 10 is a perspective view of another exemplary hinged cap device provided in accordance with aspects of the present invention; and

FIG. 11 is a perspective view of the hinged cap device of FIG. 10 in an unlocked position, showing the outer shell and the cap rotated away from the needle huh to expose the needle holder.

### DETAILED DESCRIPTION

The detailed description set forth below in connection with the appended drawings is intended as a description of exemplary embodiments of a hinged cap device for use with needles having sharp needle tips provided in accordance with aspects of the present invention and is not intended to represent the only forms in which the present invention may be constructed or used. The description sets forth the features and the steps for constructing and using the hinged cap device of the present invention in connection with the illustrated embodiments. It is to be understood that the same or equivalent functions and structures may be accomplished by different embodiments and are also intended to be encompassed within the spirit and scope of the present invention, especially those incorporating a combination of features shown in the different embodiments included herein. As denoted elsewhere herein, like element numbers are intended to indicate like or similar elements or features.

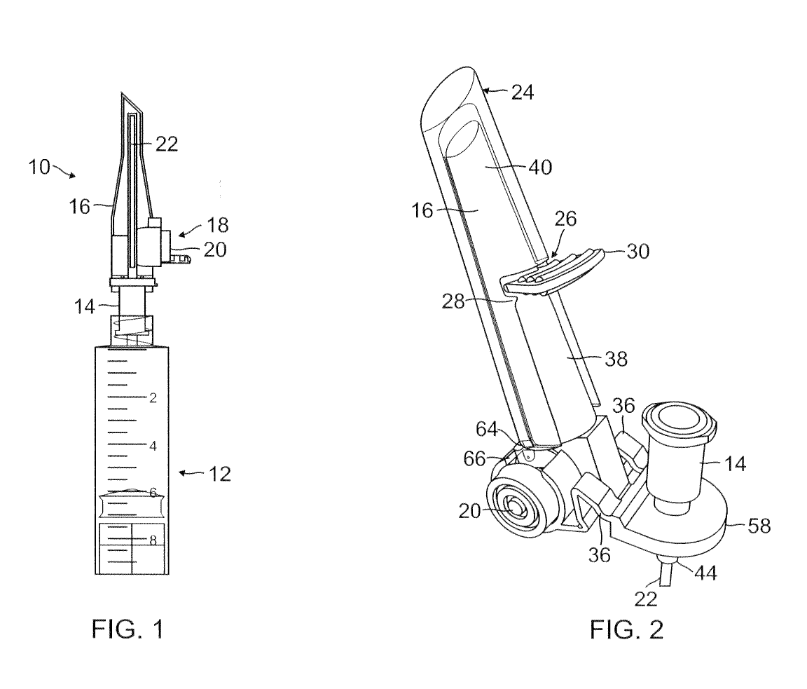

Referring now to FIG. 1, a side view of a hinged cap device or assembly 10 provided in accordance with aspects of the present invention is shown mounted on a syringe 12. As further discussed below with other figures to be described, the hinged cap device 10 comprises a needle base or needle hub 14 for mounting onto a syringe tip (not shown) and a cap, sleeve, or inner shell 16 connected to the needle hub 14 by way of a living hinge (not shown). The hinged cap assembly 10 incorporates both a temporary lock and a lock mechanism 18 for locking the cap so that it does not rotate relative to the needle hub, as further discussed below. In one embodiment, the lock mechanism 18 is a permanent lock comprising a push button or button lock 20 configured for use with a lock plate, sometimes referred to as a strike plate or surface. As will be described in more detail below, the push button or button lock 20 is depressible to lock the cap to then shield a needle 22 so that the needle is not displaceable from the cap 16 to pose needle stick injuries.

As previously alluded to, hinged cap devices should provide both a temporary lock for use during packaging and transporting between stations and a more permanent lock for locking the needle following use to prevent accidental needle sticks with a contaminated needle. With reference now to FIGS. 2, 3 and 4, in one exemplary embodiment, the hinged cap assembly 10 further comprises an outer shell, cap, or sleeve 24 positioned over the inner cap 16 for providing a temporary lock function. First regarding the inner cap 16, as shown in FIG. 4, the inner cap comprises a first sidewall 46a, a second side wall 46b, and a base wall 47 defining an elongated, generally U-shaped channel 48 therebetween. Although a square end may be incorporated, in the present embodiment, a rounded end 53 is incorporated to facilitate insertion into the outer shell 24. A gripping lever 30, located on an exterior surface 49 of the base wall 47, serves as a latching point for the outer shell 24 and also provides a handle for moving the cap relative to the needle hub 14. The cap 16 is tapered in that the diameter of a proximal portion 38 is wider than the diameter of a distal portion 40 (FIG. 2) and corresponds with the taper on the outer cap 24 for a tapered fit. However, in one embodiment, the channel 48 within the cap 16 has a constant gap or width along its length although the channel can also taper wider in the proximal direction to correspond with the cap configuration. This tapered configuration of the cap 16 allows efficient use of materials but also enhances aesthetic appeal. However, one of ordinary skill in the art will appreciate that other configurations or shape of a cap may be used without deviating from the spirit and scope of the present invention, such as making the cap generally cylindrical.

In one exemplary embodiment, the outer shell 24 is made from a transparent thermoplastic material or a semi-opaque material to allow viewing of the inner cap. The outer shell 24 may be positioned over the inner cap in a similar manner as placing a pen cap over a pen, with the requirement that the two be aligned when capped. In the present embodiment, the outer shell 24 incorporates a channel 25 that extends partially along the length of the outer cap, which in one embodiment is about half-way along the outer cap. The channel 25 on the outer shell 24 should align with the gripping lever 30 to ensure appropriate capping of the outer shell onto the inner cap. As further discussed below, the gripping lever 30 is also used to latch against a narrowed section of the channel 25 to retain the outer shell 24 to the inner cap in a detent engagement, to provide a temporary lock function,

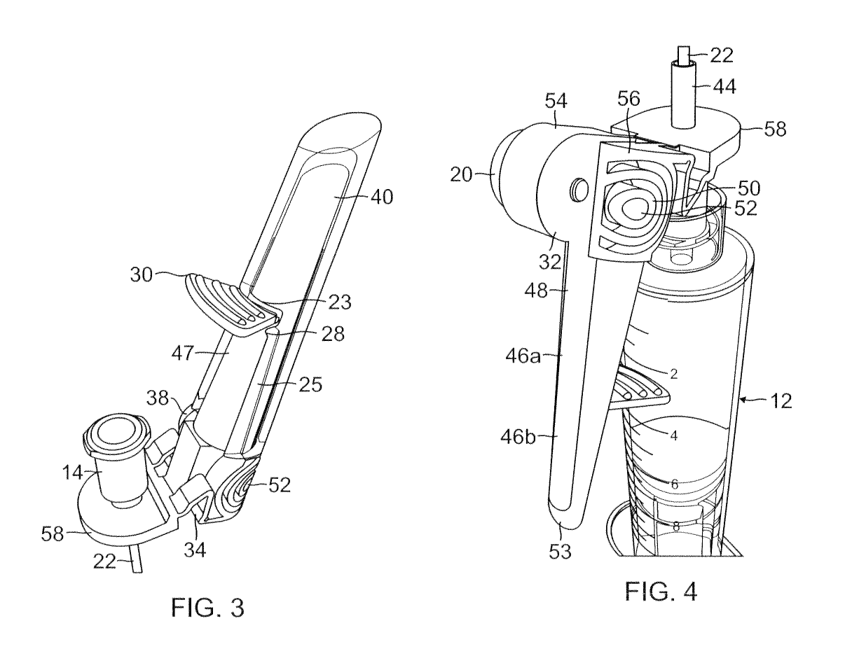

To facilitate removal of the outer shell 24 from the inner cap 16, as further discussed below, the outer shell 24 is dimensioned such that it fits snuggly over the inner cap 16. The outer shell 24 follows the contour of the inner cap 16 and covers it, except along some of the exterior surface 49 of the base wall 47, where the gripping lever 30 is located. On that exterior surface as shown in FIG. 3, the outer shell 24 covers the cap in a distal direction. However, the channel 25 extending proximally leaves the cap exposed below or proximally of the gripping lever 30.

As shown, the channel 25 has two generally parallel channel edges having a projecting formed on each edge to provide a notch 28 for providing a somewhat narrowed section on the channel to create an interference with the gripping lever. Thus, in the position shown in FIG. 3, the gripping lever 30 is bounded by the end edge 23 of the channel 25 and the notch 28, with projections formed on each side edge of the channel curving or projecting under the gripping lever 30 to fix it between the end edge and the notch 28 to thus secure the outer shell 24 to the cap 16. When the outer shell 24 is positioned on the inner cap, the needle 22 is temporary locked within the inner cap 16 as the channel 48 on the inner cap is covered by the outer shell and therefore prevents the inner cap from rotating relative to the needle. Thus, the outer shell 24 provides a temporary lock 26 for shielding the needle 22 within the cap 16. In one exemplary embodiment, the engagement between the notch 28 and the gripping lever 30 is a frictional engagement. In another embodiment, the engagement between the notch 28 and the gripping lever 30 is an interference fit. However, one of ordinary skill in the art will appreciate that toothed surfaces or other means may be used without deviating from the spirit and scope of the present invention.

The operation of the temporary lock on the hinged cap device or assembly 10 will now be described with reference to FIGS. 2-4. Initially, the device 10 is packaged with the outer shell 24 positioned over the cap 16, such that the notch 28 of the outer shell latched onto the gripping lever 30 of the cap, thus temporarily shielding the needle 22 within the cap. As used herein, temporary shielding or temporary locking is understood to mean that the needle, by one or more means, may be subsequently released from the inner cap and the inner cap pivotable away from the needle. To expose the needle 22 for use, such as to fill the syringe with medication or to administer an injection to a patient, the outer shell 24 is taken off the cap 16 by unlatching the gripping lever 30 from notch 28 and pulling the outer shell 24 in an upward direction, axially in the distal direction. Given the snug tapered fit between the outer shell 24 and the cap 16, the outer shell 24 should easily come off of the cap 16. The gripping lever 30 may then be used to move the cap 16 radially outwardly away from the needle hub 14 to expose the needle 22 for use (FIG. 4).

In one embodiment, the cap 16 remains in the “open” position away from the needle hub 14 and the needle in the ready to inject position by the bias action of a living hinge 34, which is shown in FIG. 2 as having a pair of tabs 36 extending from the needle hub 14 and connecting to the inner cap. While a gap or a void is provided to define two spaced apart tabs, the living hinge may contain only one tab or multiple tabs and it is configured to have a self bias by sizing the tabs with sufficient materials and resiliency so that it tends to push the cap and the needle hub away from one another. The shape and size of the tabs 36 determine, at least in part, how far the cap 16 is rotatable away from the needle hub 14. In other embodiments, a hook or a notch is incorporated on the cap and another on the needle hub to permit engagement between the two to retain the cap in the open position. It should be noted that although FIGS. 2 and 3 are shown with the needle holder 44 and needle 22 (partially shown) exposed and the outer shell 24 still attached to the cap 16. In practice, in order to expose the needle 22, the outer shell 24 should be separated from the cap 16 first, before the inner cap 16 can be moved radially outwardly away from the needle hub 14 to expose the needle 22.

After filling the syringe with medication and before administering the medication to a patient, for safety purposes, the needle 22 is often shielded to prevent unintentional needle sticks during transportation from between the filling station and the patient's room. As such, the temporary lock should be reactivated. For this purpose, the cap 16 is rotated radially towards the needle hub 14 to capture the needle. In one embodiment, the proximal end of the cap 16 is generally flat or planar for abutting a flange 58 located proximally of the needle holder 44 (FIG. 4) to delimit over rotation of the inner cap 16 in moving to the closed position. The flange 48 is preferably dimensioned with an outer perimeter that matches the outer perimeter on the proximal end of the cap to provide an overall smooth or aesthetic look. The outer shell 24 is then slid over the cap 16 until the notch 28 on the outer shell latches onto the gripping lever 30 of the cap 16, thus temporarily locking the needle 22 from being exposed. It may be necessary to hold the inner cap 16 over the needle while putting the outer shell 24 on due to the biasing nature of the living hinge 34.

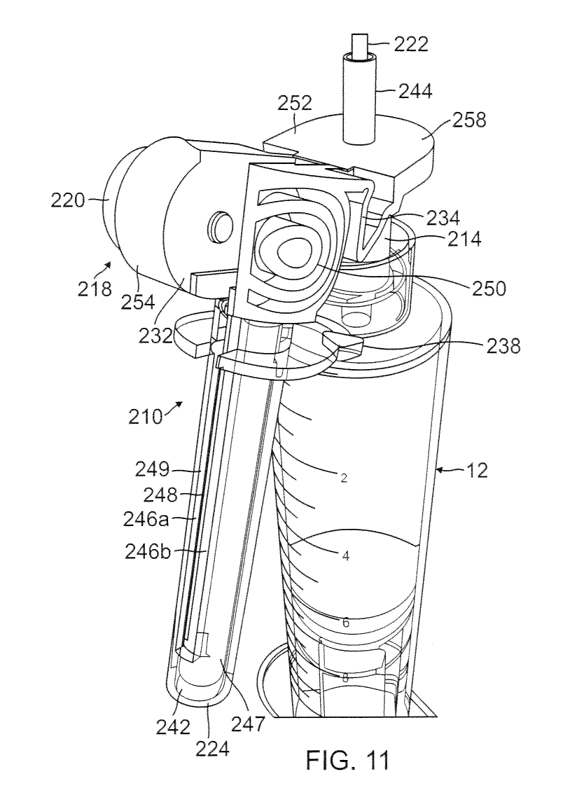

After an injection, the permanent lock 18 may be activated to more permanently capture the needle 22 within the inner cap. As shown in FIG. 4, the permanent lock 18, comprises, on the first side wall 46a, a push button 20 and, on the opposite second side wall 46b, a lock receiving plate 50, which comprises a plurality of arcuate ribs 56 to reinforce the lock receiving plate as well as add to the aesthetic appeal of the cap. In one embodiment, the ribs comprise a plurality of spaced annular or arcuate ribs (more clearly shown in FIG. 11). The ribs also increase in height as they extend from the edge of the second side wall 46b toward the center of the sidewall and then decrease in height as they extend toward the base wall.

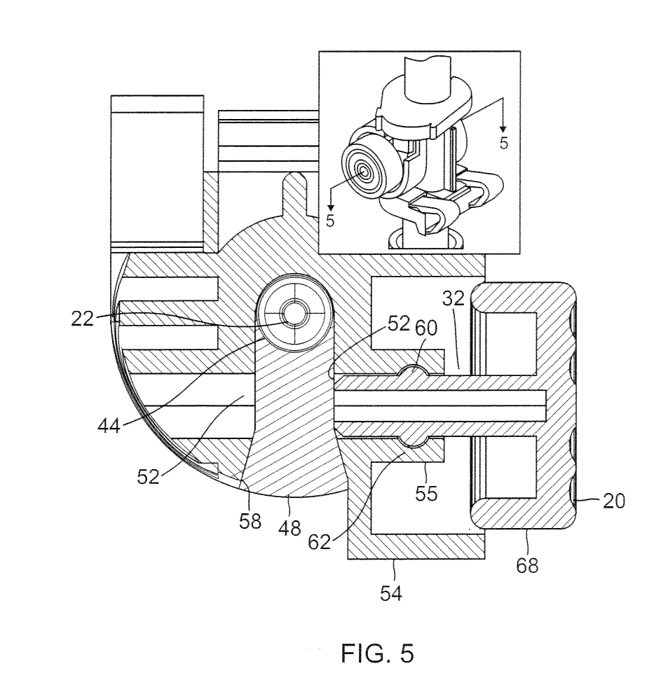

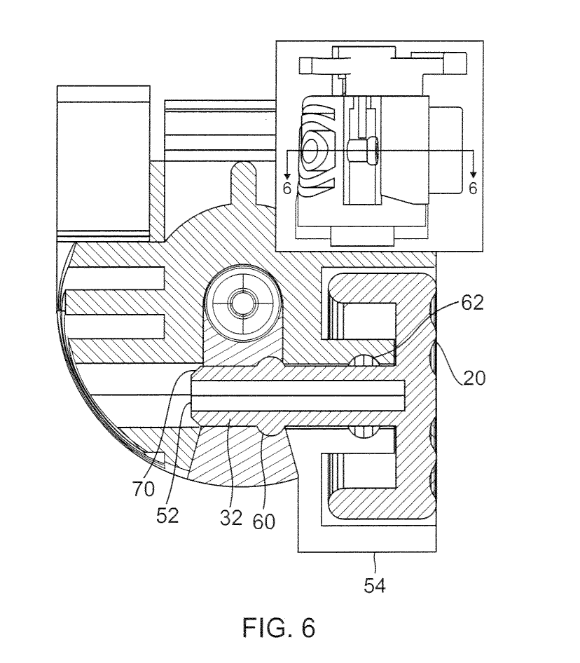

In one embodiment, the button lock or push button 20 comprises a pin 32 that projects from the first side wall 46a towards the second side wall 46h of the cap 16. The pin 32, when the push button is pushed, is engageable with a locking bore 52 located within the lock receiving plate 50 to more permanently capture the needle within the inner cap. In effect, the pin provides a barrier or obstruction to the open channel 48 to prevent the needle, and more specifically the needle holder 44, from passing back out the channel. The engagement between the pin 32 and the locking bore 52 may be implemented using interfering surfaces, as will be discussed in more detail with reference to FIGS. 5 and 6. The permanent lock 18 preferably comprises an annular well 54 for receiving the push button 20 to prevent manipulation of the same after activation, as further described below.

FIGS. 5 and 6 show cross-sectional top views of the permanent lock 18 taken along a plane drawn though the center of the push button 20 and the pin 32 while the inner cap 16 is in a vertical or closed position. In one embodiment, a first detent, male detent, or bulge 60, which resembles a half-cylinder, is formed on the shaft of the pin 32 and engages a second detent, female detent, or groove 62 formed on an inner annular well 55, which sits concentrically of the outer annular well 54. When the permanent lock is inactive (or not activated), the push button 20 protrudes from the outer well 54 and the pin 32 is kept fixed in position relative to the inner annular well 55 by the engagement between the male detent 60 and the female detent 62, as shown in FIG. 5.

To prevent unintentional or premature activation of the permanent lock 18, a safety tab 64 is incorporated at the proximal end of the outer shell 24 and is configured to project through a gap located between the annular well 54 and the flange 58 (FIG. 2). The safety tab 64 provides a barrier to the skirt 68 on the push button 20 (FIG. 5). Thus, when the outer shell 24 is positioned over the cap 16 and provides a temporary lock to shield the needle 22 within the cap 16, the safety tab 64 prevents the button lock 20 from being pushed. Therefore, to activate the permanent lock, the outer shell 24 is first removed, the inner cap 16 rotated over the needle to capture the needle 22, and then pressure is applied on the push button 20 to separate the male detent 60 from the female detent 62.

As shown in FIG. 6, pressure applied on the push button 20 by the user disengages the male detent 60 from the female detent 62, pushing the pin 32 forward. The pin is moved until the push button 20 contacts the inner annular well 55, which stops further movement of both the push button and the pin. Concurrently, the male detent 60 is moved so that it is now outside the locking bore 52 and comes to rest against the perimeter of the locking bore 52. The relative cross-sectional dimensions of the pin at the male detent 60 and the locking bore 52 prevent the pin from moving back to its original position. However, because the bore is plastic, it easily deforms when pressure is applied during button activation. In one embodiment, the pin is sized sufficiently long so that when the push button 20 is activated, the tip of the pin 70, which preferably includes a rounded taper, projects into the bore 52 on the lock plate to support the pin. In one exemplary embodiment, interference between the pin 32 and the locking bore 52 prevents the pin from moving back to its original position. However, one of ordinary skill in the art will appreciate that other means for accomplishing the same function may also be used without deviating from the spirit and scope of the present invention, such as providing a detent or a lock between the push button and the outer annular well to prevent separation between the two after activation.

Thus aspects of the present invention include a hinged cap assembly having an inner cap and an outer cap and wherein the outer cap is removable from the inner cap to then permit locking by pushing a push button. In another aspect of the present invention, a hinged cap device is provided in which a circular push button projects radially of a channel and a pin, which is attached to the push button, is configured to extend the width of the channel to obstruct the channel from ingress or egress. In yet another aspect of the present invention, an annular well is provided on a hinged cap device for surrounding a push button. Said push button having a lower lip surrounded by the annular well for preventing manipulation of the button.

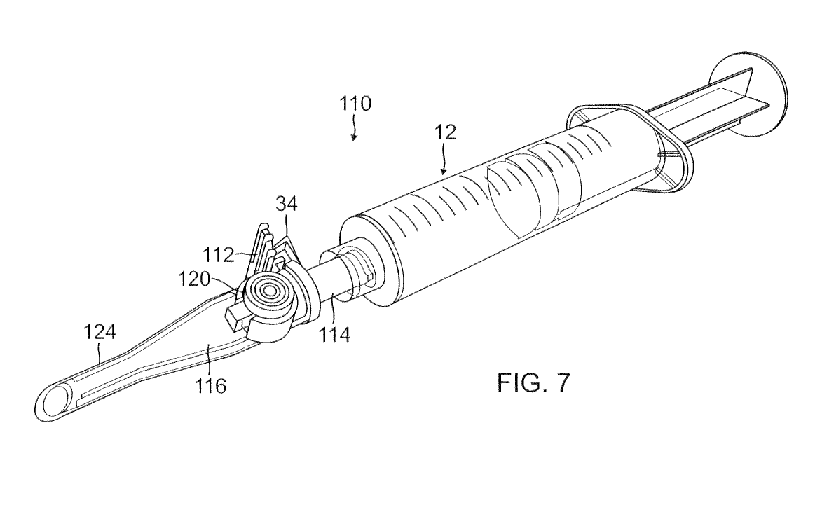

A hinged cap assembly 110 provided in accordance with an alternative aspect of the present invention is provided with reference to FIG. 7. The hinged cap assembly or device 110 comprises a needle hub 114 connected to a cap 116 via a living hinge, all having similar structures as those described above with respect to previous embodiments. Similarly, the hinged cap assembly 110 incorporates both a temporary lock and a permanent lock 118, which in the figure shown includes a push button 120. The hinged cap device 110 further comprises a removable outer shell 124 positioned over the inner cap 116 to provide a temporary lock. A gripping lever 112 is incorporated at the proximal end of the inner cap 116 to provide leverage for opening and/or closing the cap 116.

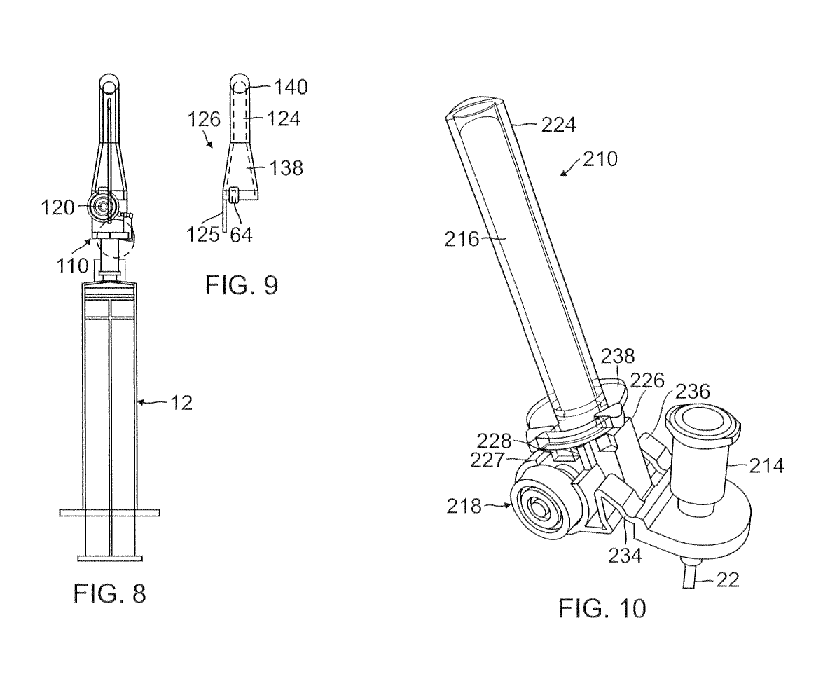

FIG. 8 shows a side view of the hinged cap device 110 of FIG. 7 while FIG. 9 shows a side view of the outer shell 124 removed from the inner cap. As shown in FIG. 9, the outer shell 124 is dimensioned to fit over the cap 116, and is gradually tapered from a wider proximal section 138 to a more narrowed distal section 140, as previously discussed with reference to the first embodiment. In the present embodiment, a tail 125 is provided at the proximal end of the cap, adjacent the safety tab 64 to prevent unintended or undesired early triggering. The tail may embody a round configuration or a multi-sided configuration, such as a square cross-section. The tail is located a finite distance from the safety tab 64 and the two define a gap therebetween. The combination tail 125 and safety tab 64 operates to grip a portion of the annular well on the permanent lock, in between the gap, to secure the two together during transporting or packaging. Thus, the outer shell 124 provides a temporary lock 126 for shielding the needle 22 within the cap 16 and incorporates a gripping means to prevent accidental displacement of the outer shell from the inner cap.

Initially, the device 110 is packaged with the outer shell 124 attached to the cap 116. To use the syringe, for example to fill the syringe with medication, a user first pulls the outer shell 124 in the upward, distal, direction to remove it from the cap 116. Then either by griping the inner cap 116 or more preferably the gripping lever 112, rotate the cap 116 away from the needle hub 114 to expose the needle (not shown) for use. To shield the needle after filling the syringe with medication and prior to administration, the temporary lock is used by first rotating the cap 116 over the needle hub 114 to capture the needle. The outer shell is then slid over the cap 116 until the outer shell bottoms out over the inner cap. Concurrently, the safety tab and the tail should be aligned to receive part of the annular well therebetween to engage the outer shell and the inner cap in a friction fit.

After administration of the medication and before safely disposing of the used syringe, the permanent lock 120 is activated by rotating the cap 116 back over the needle hub 114 to capture the needle within the cap 116. Digital pressure is then applied to the push button 120 to move the pin, as discussed above with reference to FIGS. 5 and 6.

FIGS. 10 and 11 show another exemplary hinged cap assembly 210 in accordance with aspects of the present invention. The hinged cap assembly 210 comprises a needle hub 214 connected to a cap 216 via a living hinge 234, all having similar structures as those described above with respect to previous embodiments. In one exemplary embodiment, the cap 216 comprises a generally U-shaped channel 248 defined by two side walls 246a and 246b, a base wall 247, and a rounded end 242. The cap 216 is shown in FIG. 11 with a uniform cylinder but can alternatively be tapered as previously discussed. Thus, while the cap is described as having two side walls and a base wall 247 located therebetween, the three walls may blend together, such as the case with a round cylinder. Similarly to previously described embodiments, the hinged cap assembly or device 210 incorporates both a temporary lock and a permanent lock 218, which includes a push button 220, but in a slightly different configuration.

In the embodiment shown, the hinged cap assembly 210 incorporates an outer shell or sleeve 224 positioned over the exterior of the inner cap 216. However, rather than being removable from the inner cap, the outer shell 224 comprises a lengthwise slot defining a channel 249 that is alignable with the channel 248 of the inner cap 216. The two channels 248 and 249, when aligned, are dimensioned to receive the needle 222 when the combination inner and outer caps are pivoted about the living hinge.

In one embodiment, the outer sleeve 224 is sized to form a size-on-size snug fit over the inner cap 216. The outer sleeve 224 is positioned over the inner cap and is rotatable relative to the inner cap. Both sleeves can be pivoted away together from the needle hub. Thus, when the outer sleeve is rotated so that the two channels 248, 249 are aligned with one another, the cap 216 and the outer shell 224 are displaceable/rotatable from the needle hub 214 to expose the needle 222 (FIG. 11). Conversely, when the two channels 248 and 249 are misaligned relative to one another (FIG. 10), the path for passing the needle through the cap is obstructed, thus providing a temporary lock for the needle. Accordingly, the alignment and misalignment of the two channels 248, 249 provide a temporary lock for the device 210.

To prevent relative axial movement between the inner cap 216 and the outer shell 224, a detent is incorporated between the cap and the sleeve. For example, an internal projection may be formed on the outer sleeve to mate with an external groove formed on the inner cap. As another example, a pin or rod with a ratchet tooth may be incorporated on the rounded end of the outer sleeve to project into a receiving bore formed on the inner cap to retain the two in fixed axial movement. Optionally, a visual aid or indicia 238 may be incorporated on the outer shell to facilitate rotation of the outer shell. In one embodiment, an arrow is formed on a flange along with markings to indicate the direction of rotation of the outer shell.

Initially, the device 210 is packaged with the outer shell 224 and cap 216 misaligned to secure the needle 222 within the cap 216. To fill the syringe with medication, the outer shell 224 is rotated relative to the inner cap 216 so that the outer channel 249 aligns with the inner channel 248 of the inner cap. With the two channels aligned, the outer shell 224 and the cap 216 are now pivotable radially away from the needle hub 214 to expose the needle 222. The outer shell 224 and the cap 216 may be kept in the exposed position away from the needle hub 214 by the bias characteristic of the living hinge 234, which is shown in FIG. 10 as having a pair of tabs 236 extending from the needle hub 214. Alternatively, however, the living hinge may embody a single tab or multiple tabs and configured to be self biasing by sizing the tabs with sufficient materials and resiliency so that it pushes the cap and the outer shell away from the needle hub. Alternatively, a latching feature may be used to retain the cap in the open position, as previously discussed.

To activate the temporary lock to shield the needle 222 within the cap 216, for example after filling the syringe with medication and prior to administration to a patient, a user moves the outer shell 224 and the cap 216 radially inwardly towards the needle hub 214 until the needle 222 is captured within the cap 216. The outer shell 224 is then rotated relative to the cap 216 so that the channels 248 and 249 become misaligned, thus securing the needle 222 within the cap.

After administering medication to a patient, the needle 222 may be permanently and safely secured within the cap by activating the permanent lock 218 located at the proximal end of the cap 216. As previously described for other embodiments, for example with reference to FIGS. 5 and 6, the push button 220 comprises a pin which upon activation engages a locking bore 252 located within a lock receiving plate 250 (FIG. 11).

In one embodiment, a tab 228 extending axially at the proximal end of the outer shield 224, proximally of the flange 238, is incorporated to abut the push button 220 in the packaged and the ready-to-use position to prevent the push button 220 from being depressed unintentionally in these positions. The tab 228 is also configured to abut a shoulder 226 of the lock to limit rotation in one direction and a part or edge 227 of the annular wall to limit rotation in the other direction.

To permanently secure the needle 222 for safe disposal and prevent needle stick, as previously described for previous embodiments, digital pressure is applied on the push button 220 to move the pin into engagement with the locking bore. The interactions between different components of the permanent lock 218 are similar to those described previously with reference to FIGS. 5 and 6. Briefly, when the permanent lock 218 is inactive, the push button 220 projects away from the annular well. The pin 232 is kept fixed in position within the annular well 254 by detent engagement, similar in structures to the male detent 60 and female detent 62 described with reference to FIGS. 5 and 6. The applied digital pressure on the push button 220 uncouples the detent and allows the pin 232 to be displaced into the locking bore 252 located within the lock receiving plate 250.

Thus, aspects of the present invention include a hinge cap device comprising a rotatable element for providing a temporary lock and a pushable pin for providing a permanent lock. Another aspect of the present invention is a push button located on one end of the pin, the push button being surrounded by an annular well for covering, at least in part, the push button following activation. In a still further aspect of the present invention, a rotatable flange is provided on an outer shell, said rotatable flange comprising instruction indicia and a tab for limiting rotation.

Although limited hinged cap embodiments and their components have been specifically described and illustrated, many modifications, combinations, and variations of the embodiments will be apparent to those skilled in the art. For example, the length, size, colors, and other appearances of the cap and outer shell may be modified, and the needle may be attached directly to the hub on the hinged cap or as a separate hub attached via a luer fitting. Furthermore, it is understood and contemplated that features specifically discussed for one hinged cap embodiment may be adopted for inclusion with another hinged cap embodiment provided the functions are compatible. Accordingly, it is to be understood that the hinged cap devices and their components constructed according to principles of this invention may be embodied other than as specifically described herein. The invention is also defined in the following claims.

What is claimed is:

1. A hinged cap device comprising a hub having a needle attached thereto and a cap hingedly connected relative to the hub; wherein the cap comprises a space for receiving the needle in a secured position.

2. A hinged cap device for use with a syringe comprising:
a needle hub comprising a cavity for fitting onto a syringe tip, said needle hub comprising a needle comprising a needle shaft and a needle tip;
a cap defining a channel for capturing the needle to shield the needle tip;
a living hinge connecting the cap to the needle hub;
a depressable button lock for locking the needle so that the needle is not displacable from the cap; said depressable button lock being located on a proximal end of a first side wall of said cap and comprising a pin projecting from said first side wall towards a second side wall of said cap; and
a lock receiving plate located on said second side wall of said cap at a location for receiving said pin.

3. The hinged cap device of claim 2, wherein the living hinge comprises a pair of tabs extending from the needle hub and having a gap therebetween.

4. The hinged cap device of claim 2, wherein the cap comprises a gripping lever.

5. The hinged cap device of claim 2, wherein the lock receiving plate further comprises arcuate ribs extending away from the second side wall.

6. The hinged cap device of claim 2, wherein the needle hub further comprises a flange comprising a planar surface for contacting a proximal surface of the cap.

7. The hinged cap device of claim 2, further comprising an outer shell positioned over the cap.

8. The hinged cap device of claim 7, wherein the outer shell is removable from the cap.

9. The hinged cap device of claim 7, wherein the outer shell comprises a lengthwise slot defining a channel that is alignable with the channel of the cap to permit rotation of the cap and the outer shell away from the needle.

10. The hinged cap device of claim 7, wherein the outer shell further comprises a safety tab which is movable away from the depressable push button lock to allow the pin to be engageable within the lock receiving plate.

11. A hinged cap device for use with a syringe comprising:
a needle hub comprising a cavity for fitting onto a syringe tip, said needle hub comprising a needle comprising a needle shaft and a needle tip;
a cap defining a channel for capturing the needle to shield the needle tip;
a living hinge connecting the cap to the needle hub;
a locking mechanism on the cap for securing the needle within the cap, the locking mechanism comprising a pin projected from a button lock located on a first side wall of said cap;
a locking bore located on a second side wall of said cap; and
wherein the pin is engageable within the locking bore to secure the needle within the cap.

12. The hinge cap device of claim 11, wherein the living hinge comprises a pair of tabs extending from the needle hub and having a gap therebetween.

13. The hinged cap device of claim 11, further comprising an outer shell positioned over the cap.

14. The hinged cap device of claim 11, wherein the in is engageable within the locking bore by interference fit.

15. The hinged cap device of claim 11, wherein the needle hub further comprises a flange extending radially of the hub and defining a planar surface for contacting a proximal surface of the cap.

16. The hinged cap device of claim 13, wherein the outer shell is removable from the cap.

17. The hinged cap device of claim 13, wherein the cap further comprises a gripping lever.

18. The hinged cap device of claim 13, wherein the outer shell further comprises a groove along a lengthwise axis and a channel.

19. The hinged cap device of claim 11, wherein the locking mechanism further comprises a safety tab which is movable to allow the pin to be engageable within the lock bore.

20. A method for manufacturing a hinged cap device for use with a syringe, said method comprising:
forming a needle hub having a cavity for mounting on a syringe tip and attaching a needle to the needle hub;
forming a primary cap having a base wall, a first side wall, and a second side wall, which together define a channel, and attaching the primary cap to the needle hub with a living hinge;
rotating the primary cap so that the needle is captured within the channel defined by the primary cap;
providing a temporary lock so that when the temporary lock is activated, the primary cap is temporarily blocked from being rotated away from the needle;
providing a permanent lock so that when the permanent lock is activated, the primary cap is permanently blocked from being rotated away from the needle; and
wherein the permanent lock is located on one of the two side walls and comprises an annular race defining a cavity, a push button comprising a push wall comprising a push surface movable into the cavity of the annular race, and a pin extending from the push wall and configured to extend across the channel of the primary cap when the permanent lock is activated to contact a lock receiving plate on the second side wall.

21. The method of claim 20, further comprising the step of providing an outer shell exterior to the primary cap.

22. The method of claim 21, wherein the temporary lock is provided on the outer shell.

23. The method of claim 21, wherein the outer shell is removable from the primary cap.

24. The method of claim 21, wherein the outer shell comprises a groove along a lengthwise axis and a channel.

25. The method of claim 20, wherein the permanent lock is activated by moving the pin into the lock receiving plate and providing an interference fit to prevent separation of the pin from the lock receiving plate.

26. The method of claim 20, wherein the permanent lock further comprises a safety tab which is movable to allow the pin to contact the lock receiving plate.
